(19) United States
(12) Patent Application Publication
(10) Pub. No.: US 2022/0330949 A1
(43) Pub. Date: Oct. 20, 2022

(54) COMPRESSION BALLOON WITH A RIGID FOOT FOR HEMOSTASIS

(71) Applicant: Biolife, L.L.C., Sarasota, FL (US)

(72) Inventor: Talmadge Kelly Keene, Ruskin, FL (US)

(21) Appl. No.: 17/724,318

(22) Filed: Apr. 19, 2022

Related U.S. Application Data
(60) Provisional application No. 63/176,427, filed on Apr. 19, 2021.

(51) Int. Cl. A61B 17/135; A61B 17/132

(52) U.S. Cl. CPC A61B 17/135; A61B 17/1325; A61B 2017/12004

(57) ABSTRACT

This device is a combination of an expandable or inflatable bladder or balloon in conjunction with a rigid or semi-rigid skin contact surface or structure to reduce bleeding from a skin insertion wound site and a vascular wound site after a vascular intervention or surgical procedure is performed. The device may be held in contact or close contact with the skin by a circumferential band, a clamp or other mechanism and provides a more consistent and uniform surface area contact and compression force. The contact surface or foot provides a uniform surface area contact as compared to the ever-changing contact surface area from an inflatable bladder.

### RELATED APPLICATION

The present application claims the benefit of U.S. Provisional Patent Application No. 63/176,427, filed on Apr. 19, 2021, the entire disclosure of which is incorporated by reference herein.

### FIELD OF THE INVENTION

The present invention relates in general to hemostasis, methods of achieving same, devices used in such methods, and, in particular, to hemostasis of a blood vessel immediately after vascular catheterization procedure.

### BACKGROUND OF THE INVENTION

Many medical procedures that once required extensive invasive surgery are performed today less evasively by inserting surgical or diagnostic devices through arteries or veins (i.e., vascular procedures). These procedures are much safer and require significantly less recovery time. To prevent clots from forming in the vessels after surgery, the patient may require anticoagulation medications, which often results in excessive bleeding. To stop bleeding after vascular procedures, most clinics use direct pressure applied directly to the wound. This pressure must be held over both the entry point wound in the skin and the wound that was created in the vessel.

Devices exist that are designed to be inserted into the patient to directly close the hole in the vessel, but such devices are expensive. Moreover, the devices must remain in the patient and they prevent reuse of the vessel for some time. Therefore, most clinics continue to use direct pressure to stop the bleeding.

Direct pressure can be applied manually by a clinician, but in many instances the pressure is needed for an extended period of time to decrease or stop the bleeding. To save the clinicians time, many compression devices have been invented. Many such devices employ an inflatable bladder to create the compression needed to stop the bleeding. This compression must, again, cover the wound in the skin as well as the wound in the vessel beneath the skin.

Recent testing has revealed that the surface area created by an expandable bladder/balloon may be significantly different as the balloon is inflated or deflated. This surface area will vary both with inflation, and with manual compression force applied to the balloon. More inflation will create a smaller surface area as the balloon tends to round. More manual compression will tend to increase the surface area as the balloon is flattened.

The present invention addresses one or more of these long-felt needs to allow for a more constant surface area, resulting in a more consistent uniform pressure that is not possible with current compression bladders/balloons.

### SUMMARY OF THE INVENTION

This invention relates generally to using a rigid or semi-rigid device having a relative constant surface area to cover both the wound in the skin and wound in the artery beneath an inflatable bladder or balloon to improve hemostasis after a surgical procedure, such as a vascular access procedure.

Briefly, the present invention is directed, in an embodiment, to a device for creating improving hemostasis, wherein the device comprises an inflatable bladder and further comprises a rigid or semi-rigid surface or foot on the patient-side of the bladder. In using the device, the rigid or semi-rigid surface will be placed adjacent to the patient's skin and between the bladder and skin to provide a more consistent surface area over which the compression created by inflating the bladder will be applied. The use of a more rigid structure beneath the balloon (between the balloon and the skin) diminishes the surface area variability that is unavoidable with currently marketed bladders or balloons, and creates more uniform compression pressure, across the necessary area, needed to be compressed to create hemostasis at the skin and at the vessel beneath.

In another embodiment, the invention is directed to an improvement to a hemostasis device having an inflatable bladder or balloon that is applied to and held on to the patient with a circumferential wrap, wherein the improvement comprises a rigid or semi-rigid structure adjacent to the bladder or balloon and which, in use, will be applied to and held on to the patient by the wrap. The device is designed such that when the bladder is inflated it pushes on the rigid or semi-rigid structure which, in turn, pushes on the patient with a more consistent surface area force than devices not employing the rigid or semi-rigid structure. The band may be secured around the patient in any of a number of ways including but not limited to Velcro, snaps, buttons, adhesive, or a zip-tie like mechanism.

In certain embodiments, the rigid or semi-rigid structure may be adhered to the outside or bottom-side of the bladder with an adhesive such as a glue, or may be, for example, heat-sealed to the bladder.

In other embodiments, the rigid or semi-rigid structure may be adhered to the bladder at a center point.

In other embodiments, the rigid or semi-rigid structure may be adhered to the bladder at multiple points.

In other embodiments, the rigid or semi-rigid structure may be fully adhered to the bladder.

In other embodiments, the rigid or semi-rigid structure may be adhered to the inside of the proximal side of said bladder.

In other embodiments, the rigid or semi-rigid structure may be adhered by being welded to the bladder or as the bottom part of the bladder.

In other embodiments, the rigid or semi-rigid structure may be adhered to the bladder through an attachment mechanism on the bladder such as a pocket or a male/female snap-in-place attachment mechanism.

In other embodiments, the device may be designed to allow for multiple rigid or semi-rigid structures, which may vary in size, to be connected or otherwise adhered to the bladder.

In yet another embodiment, an improved hemostasis device comprises a bladder or balloon and a rigid or semi-rigid structure designed such that when the bladder is inflated, it pushes on the rigid or semi-rigid structure which, in turn, pushes on the patient with a more consistent surface area and/or compression force than known similar hemostasis devices not having the rigid or semi-rigid structure. In such embodiments, the rigid or semi-rigid structure is designed to be placed between the bladder and the patient when in use.

In other embodiments, the rigid or semi-rigid structure may be a separate device that is applied to the patient before the device housing the bladder is applied to the patient.

In other embodiments, the rigid or semi-rigid structure may be adhered to the bladder prior to application to the patient.

In other embodiments, the rigid or semi-rigid structure may be inserted into a pocket on the bladder or held in place by a male/female type snapping securement.

In yet another embodiment, a device for improving hemostasis, particularly after a vascular procedure, may comprise a bladder or balloon and a rigid or semi-rigid structure. The device is designed such that when the bladder is inflated it pushes on the structure which, in turn, pushes on the patient with a more consistent surface area and the structure is integral with the bladder or balloon and forms the bottom portion of the bladder which will be in contact with the patient's skin.

In other embodiments, the bladder or balloon may be the rigid or semi-rigid structure and may be formed with gussets along its sides such that the bladder or balloon acts like a bellow, forcing expansion vertically as it is inflated.

In other embodiments, the bottom or contact surface of the bladder or balloon may be rigid or semi-rigid or the contact surface of the bladder or balloon may be composed of a material or thickness that resists balloon rounding.

### BRIEF DESCRIPTION OF THE DRAWINGS

FIG. 1 is a cross-section sketch of a typical procedure performed through vascular access in a patient's body.

FIG. 2 illustrates a known method, in cross-section, for applying pressure to a wound after a vascular procedure.

FIG. 3 illustrates another known method, in cross-section, for applying pressure to a wound after a vascular procedure.

FIG. 4 is a photograph of a simulated pressure application using one's finger after a vascular access procedure.

FIG. 5 is cross-section showing an embodiment of the present invention.

FIGS. 6A-6F are simulations of wound pressure applied with known methods.

FIGS. 7 and 8 are graphs showing the results of some of the simulations in FIGS. 6A-6F.

### DETAILED DESCRIPTION OF PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

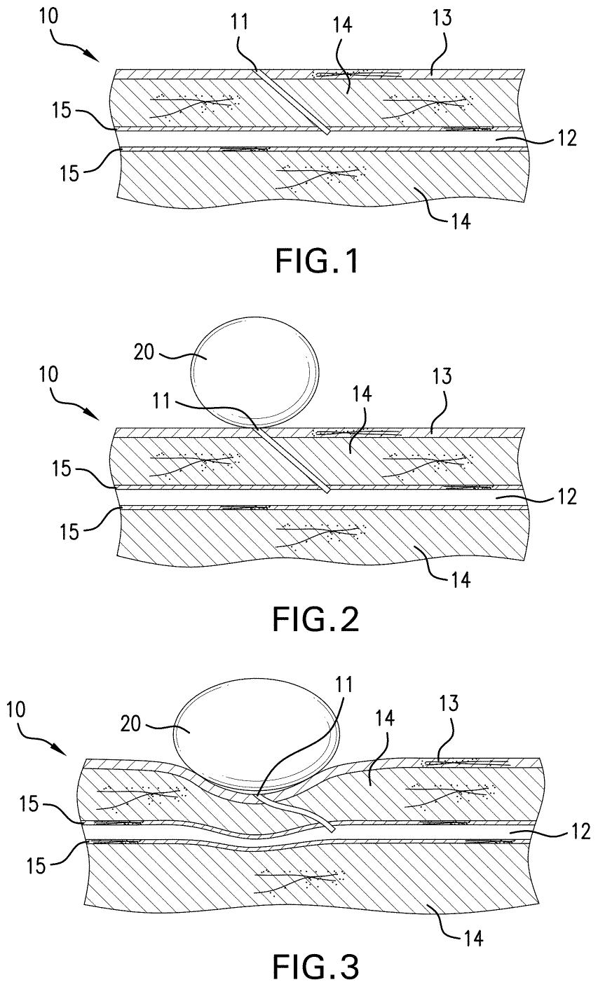

FIG. 1 shows in sketch form a typical procedure performed through vascular access in a patient's body part 10. Channel 11 is a typical insertion track into a blood vessel 12. Channel 11 is formed through skin 13, tissue 14, and blood vessel wall 15 to reach blood vessel 12.

The wound created by insertion of the instrument insertion through skin 13 (i.e., the “insertion wound”) is often not aligned vertically with the wound created through tissue 14 to reach blood vessel 12 (i.e., the “vascular wound”). This difference in vertical alignment typically increases with length of channel 11 and depth of blood vessel 12. Therefore, different sized and shaped structures may be necessary to meet the variety of medical applications. Accordingly, to stop the bleeding after performing a vascular procedure, a clinician needs to hold pressure over both the insertion wound in the skin 13 and the vascular wound in tissue 14.

A known method for applying pressure to the wound after a vascular procedure is shown in FIGS. 2 and 3. In particular, as shown in FIG. 2, an inflatable balloon 20 may be used to create compression over the wound site. As compression is applied by inflating balloon 20 as shown in FIG. 3, the balloon will flatten to apply pressure to skin 13 at the insertion wound site. However, if the insertion wound site is not sufficiently aligned vertically with the vascular wound site at blood vessel 12, balloon 20 may not apply compression over the vascular wound. This can result in a hematoma because the vascular wound in blood vessel 12 is not controlled by manual compression and may continue to bleed.

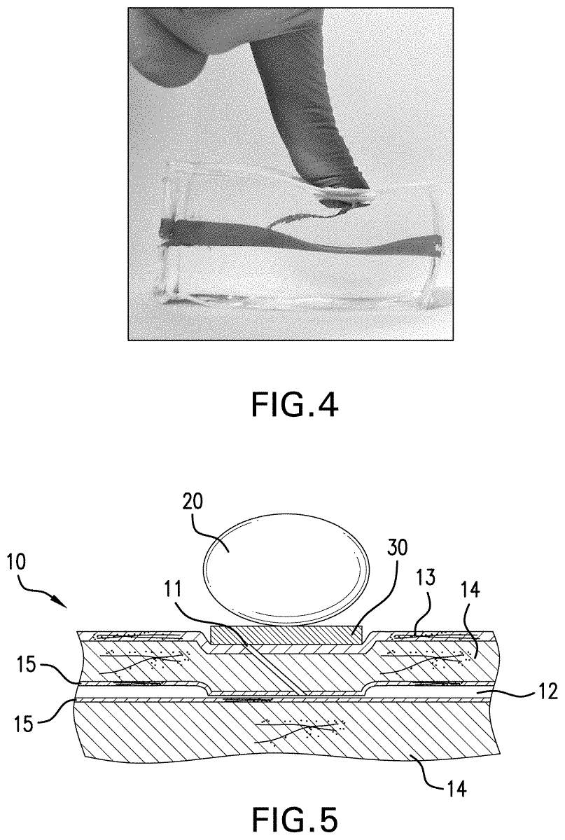

Another example of insufficient pressure at the insert wound site and the vascular wound site is shown in the photograph of FIG. 4 depicting a simulated femoral artery that was created using ballistic gel. The insertion wound site (directly beneath the finger) is controlled, but the vascular wound site in the simulated blood vessel is not adequately compressed. This would likely result in a hematoma in a clinical setting. Had an appropriately sized, rigid structure been used beneath the finger to create a constant surface area and relatively constant compression over both sites, both the insertion wound in the skin and the wound in the blood vessel would have been controlled.

However, if a rigid or semi-rigid structure 30 is used beneath the bladder or balloon 20 as shown in FIG. 5, the use of a such a structure 30 decreases the variation of the rounded balloon surface area and allows for relatively consistent compression to both the skin insertion wound site and the vascular wound site.

Use of such a device requires that the rigid or semi-rigid structure 30 (such as a plate) be positioned between the bladder/balloon 20 and the skin 13 when used. The particular method of adhering the structure to the bladder/balloon or causing its placement between the bladder/balloon without adherence to the bladder/balloon is not important, provided this positioning is maintained.

This device may be used in connection with bands designed to wrap circumferentially around a limb, or other body parts. It may also be used in connection with other mechanical means like clamps.

The size and shape of the rigid or semi-rigid structure 30 may vary depending on the specific clinical need. The structure will typically be designed to have a size such that the structure covers both the insertion wound in the skin and the wound in the vein or artery. For a radial artery procedure, the structure may, for example, be from 0.5-1.5 inches wide and 1.0-2.0 inches long. Structures used for procedures such as femoral artery procedures may need to be larger because the distance from the skin insertion wound to the artery wound will be greater, because the artery is deeper in the body, resulting in a larger mis-alignment of the skin-wound and vessel-wound. Variations from person to person could also result in varying size requirements.

The rigid or semi-rigid structure may employ different shapes, including but not limited to round, oval, square or rectangular shapes.

The rigid or semi-rigid structure may be applied separately, before the balloon device is applied to the patient. The structure may also be part of the bladder and integral therewith. The structure may be adhered to the bladder through some type of chemical (e.g., glue) or mechanical (e.g., claims) means. The structure may be adhered inside or outside the bladder and may be adhered at a single small point to provide a pivot point or it may be adhered at multiple points or across a greater amount of the surface area of the bladder/balloon.

The structure may be transparent to allow the clinician to visualize the site.

The structure may be flat or curvilinear, depending on clinical need and the design of the compression device. The structure may be encapsulated in a soft comforting material to prevent sharp edges from harming the patient.

The structure may directly contact the skin or it may have a dressing or other covering for placing between the structure and the patient's skin. The structure may incorporate a hemostatic material, antimicrobial agents, or wound benefiting medication.

Using inflatable hemostatic products today, as the balloon is inflated, it applies more compression over the wound site. Inflation increase compression over the site while simultaneously reducing the surface are over which that compression force is applied. In addition, the balloons become more rounded creating irregular pressure (pounds per square inch or mmHg) over the contact surface area. This multiplier effect with currently marketed products makes estimating pressure impossible.

Typically, balloons are inflated then the sheath or catheter is pulled through the mechanically compressed vessel. Because of the way that a round balloon applies such uneven pressure across the surface, and more inflation reduces the area, thus concentrating the compression pressure (psi) there is potential to cause severe damage to the inner walls of the vessel. The rigid foot, spreading out this pressure over a uniform surface will help to prevent this from happening.

The uniform surface will also help to facilitate standard inflation/deflation protocols. This is much less practical when using a balloon with a dynamic surface area that varies with inflation or mechanical compression.

### EXAMPLES

### Example 1

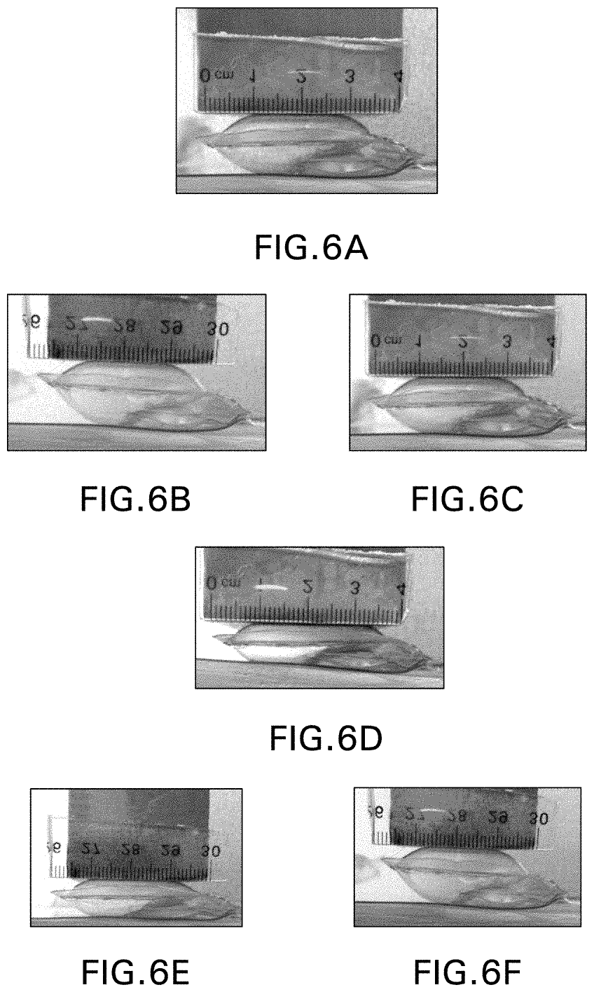

The balloon shown in FIG. 6A below is the balloon from a market-leading band used for hemostasis post-radial access procedures (TR Band—Terumo) inflated with 16 ml of air and a 1267-gram mass applied. In application, the patient's skin would be in the location of the mass with the attached measurement device.

For this example, the balloon is upside down and the patient's skin would be where the measurement device (ruler “cm”) is located. Note the area of contact between the mass and the balloon.

### Example 2

The balloons shown in FIGS. 6B, 6C, and 6D are the same balloons filled with 16 ml of air, with 3 different masses— 755 grams, 1267 grams, and 3833 grams, respectively—applied atop the balloon. Note the variation in area of contact between the different masses and the balloon with the same inflation.

### Example 3

The balloons shown in FIGS. 6E and 6F are the same balloons filled with 10 ml and 16 ml of air, respectively, with the same mass of 755 grams applied atop. Note the variation in area of contact between the same mass and the balloon with different fill volumes.

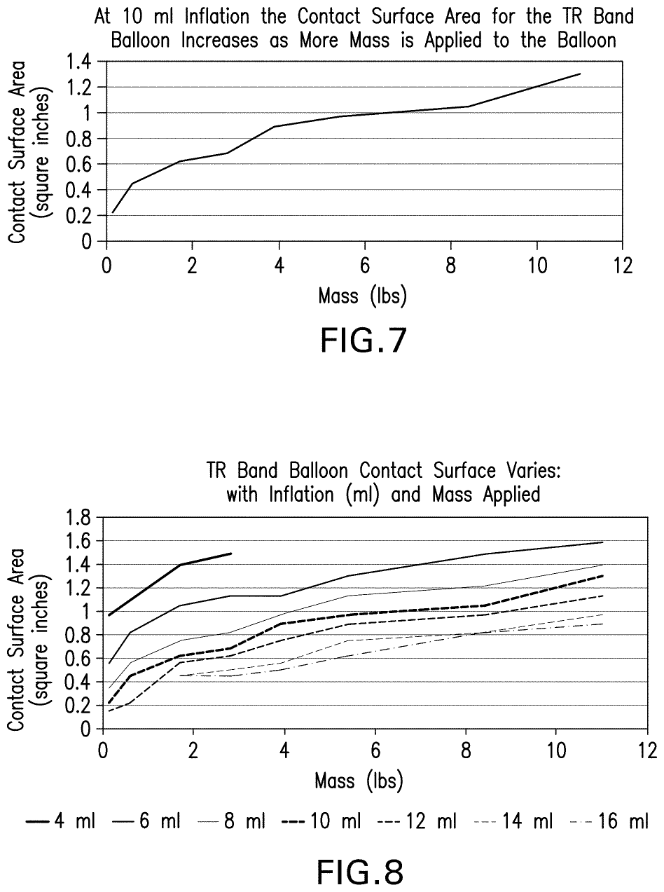

The graphs of FIGS. 7 and 8 are generated from the examples above. It is evident from these graphs that there is tremendous variation in the surface area and that surface area varies with inflation and compression. This variation will be reduced or completely removed if a rigid or semi-rigid structure is placed between the balloon and the weight (simulating a structure between a balloon and a patient). Reducing this variation will have positive clinical implications.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the versions contained therein.

What is claimed is:

1. A compression device comprising a housing, the housing comprising an expandable bladder for applying pressure to a skin wound site and a vascular wound site to assist in slowing the bleeding of both wounds, the bladder having a first side and a second side for being placed against the skin of a patient having a skin insertion wound site, the compression device further comprising a rigid or semi-rigid structure adjacent the second side of the bladder for obtaining a relatively constant surface area and compression force over the skin where the skin insertion wound site is located and where the vascular wound site is located when the compression device is applied to the patient's skin and the bladder is expanded.

2. The compression device of claim 1 wherein the housing is a circumferential wrap.

3. The compression device of claim 1 wherein the rigid or semi-rigid structure is adhered to the second side of the bladder with an adhesive.

4. The compression device of claim 1 wherein the rigid or semi-rigid structure is adhered to the second side of the bladder at a center point.

5. The compression device of claim 1 wherein the rigid or semi-rigid structure is adhered to the second side of the bladder at multiple points.

6. The compression device of claim 1 wherein the rigid or semi-rigid structure is adhered to the second side of the bladder along the entire surface of the rigid or semi-rigid structure.

7. The compression device of claim 1 wherein the bladder has a cavity that may be filled with air or other fluid, and an outer surface and an inner surface, and wherein the rigid or semi-rigid structure is housed within the bladder cavity.

8. The compression device of claim 7 wherein the rigid or semi-rigid structure is adhered to the inner surface of the bladder.

9. The compression device of claim 7 wherein the rigid or semi-rigid structure is the bladder itself such that the bladder resists rounding on the second side when the bladder is inflated.

10. The compression device of claim 1 wherein the rigid or semi-rigid structure is welded to the second side of the bladder.

11. The compression device of claim 1 wherein the housing or the bladder has a pocket and the rigid or semi-rigid structure is housed within the pocket.

12. The compression device of claim 1 wherein the rigid or semi-rigid structure is adhered to the second side of the bladder with a male/female snap-in-place attachment mechanism.

13. The compression device of claim 1 wherein the compression device comprises at least one additional rigid or semi-rigid structure.

14. A method for simultaneously applying pressure to a skin insertion wound site and a vascular wound site and causing hemostasis by:
a. applying a compression device to the skin insertion wound site and a vascular wound site, wherein the compression device comprises an expandable bladder;
b. placing a rigid or semi-rigid structure between at least a portion of the expandable bladder and the skin insertion wound site and vascular wound site; and
c. expanding the expandable bladder.

15. The method of claim 14 wherein the rigid or semi-rigid structure is adhered or attached to the bladder.

16. The method of claim 14 wherein the rigid or semi-rigid structure is integral with the bladder.

17. The compression device of claim 1 wherein the bladder has a first side and a second side and the sides of the bladder are designed to force the bladder to move vertically to form a bellow on the first side and a flatter surface on the second side when used.

18. The compression device of claim 1 wherein the bladder comprises a bladder wall thickness sufficient to force vertical expansion.

19. The compression device of claim 1 wherein the bladder comprises gussets to force vertical expansion.

20. The compression device of claim 1 comprising an adjacent rigid or semi-rigid structure wherein the first side of the bladder is adjacent to the structure to limit expansion in the direction away from the skin.

21. The compression device of claim 1 wherein both sides of the bladder are integral with the rigid or semi-rigid structure.

22. The compression device of claim 1 wherein both sides of the bladder are attached to the rigid or semi-rigid structure.

23. The compression device of claim 1 wherein both sides of the bladder are adjacent to the rigid or semi-rigid structure.

24. The compression device of claim 1 wherein the compression device is transparent.

25. The compression device of claim 1 comprises medication such as an antimicrobial, hemostatic material, or other wound treatment materials.
